(19) United States
(12) Patent Application Publication
(10) Pub. No.: US 2008/0171979 A1
(43) Pub. Date: Jul. 17, 2008

(54) Rapid exchange stent delivery catheter

(75) Inventors: Peter S. Brown, Palo Alto, CA (US); Lawrence John Voss, San Jose, CA (US); Frank Litvack, Los Angeles, CA (US)

Correspondence Address: Intellectual Property Department;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/582,521

(22) Filed: Oct. 17, 2006

(51) Int. Cl. A61M 29/00; A61M 31/00; A61M 37/00

(52) U.S. Cl. 604/103.04

(57) ABSTRACT

A rapid exchange balloon catheter has a short rapid exchange length for faster catheter exchanges the balloon catheter includes a balloon structure having a balloon leg connected to a catheter shaft. Marker bands may be provided in the balloon leg for facilitating measurement of a dimension of physiological features. A stiffening wire extending longitudinally at least partially into the balloon leg may be provided to supplement and control the flexibility characteristics of the balloon leg.

### BACKGROUND OF THE INVENTION

1. Field of the Invention

The invention relates to interventional catheters, and more particularly, a rapid exchange stent delivery catheter.

2. Brief Description of the Related Art

Stent delivery catheters are known in the art. They generally comprise an elongated tubular device having a distal portion that carries a stent for delivery to a site in a patient's vasculature. The stent may be medicated and is used to prop open and support a constricted or otherwise compromised section of the blood vessel, due for example to arterial sclerosis. The stent is delivered in a contracted state so that it can be maneuvered through the vasculature. Once it is at the desired location, it is expanded and decoupled from the catheter. The delivery catheter is then withdrawn.

The delivery catheter is provided with a balloon on which the stent is securely seated during delivery. When the stent reaches its destination, the balloon is inflated, causing the stent to expand in the lumen. The balloon is then deflated so that it disengages from the stent, allowing withdrawal of the catheter.

Stents have many different configurations and sizes, and proper selection of the stent is an integral part of the procedure. Often, the determination of whether a particular stent is suitable can only made after the stent is delivered to the lesion site. Then, if the determination is that the stent is not suitable—for example because it is of the wrong length—then before inflation the stent must be withdrawn and replaced with a different stent. Alternatively, if a stent which is too short is implanted, an additional overlapping stent may be needed. Therefore there is a long-felt need to improve the ability to pre-assess the suitability of a particular stent, before it is expanded. This task is complicated by limitations of imaging technology. In most cases, imaging or visualization of a lesion is compromised by limitations of the viewing angle because the visualization system may not be positioned to provide a direct side view of the vessel. The view in this instance is foreshortened because of the orientation of the visualization system relative to the vessel, making a determination of lesion length even more difficult. This issue is further aggravated when curved vessels are involved, making length determination even more problematic.

The rapid exchange stent delivery catheter itself is designed so that it can be quickly withdrawn and replaced if it is determined that its stent payload is of the wrong configuration for that particular application. As seen in FIG. 1, the generally tubular structure 10 defining the rapid exchange stent delivery catheter 11 is provided with a secondary tube 12 enclosed within the tubular structure. Secondary tube 12 provides a guidewire lumen which extends through the balloon 14. Balloon 14 is in fluid communication with lumen 15 of tubular structure 10 such that the balloon can be inflated or deflated as necessary. Secondary tube 12 communicates with the exterior of the catheter by way of a distal port 16 located at the tip of the catheter, and a proximal port 18 located proximally of the balloon.

With reference to FIG. 2, secondary tube 12 is designed to accommodate a guidewire 20 used to guide the catheter to the delivery site. During operation, the guidewire 20, along with a guiding catheter 22, are used to direct the rapid exchange stent delivery catheter 111 to the delivery site in the patient. When the distal end of the guidewire 20 is properly situated at the delivery site, loading of the rapid exchange stent delivery catheter 11 and introduction thereof into the patient can commence. This involves passing the proximal end of the guidewire 20, which protrudes from the patient, into the catheter's distal guidewire port 16, through secondary guidewire tube 12, and out proximal guidewire port 18. The catheter 11 is then guided along the guidewire 20, through the length of the guiding catheter 22 in the patient, and then out distal end 24 of the guiding catheter and further to the delivery site. The stent 26 is then decoupled from the balloon 14 and the delivery catheter 11 is withdrawn.

An example of a device having a configuration similar to that described above is the angioplasty-type balloon catheter described in U.S. Pat. No. 5,061,273 (Yock). In the Yock patent, it was recognized that important advantages can accrue from maintaining a transition region associated with the proximal guidewire port of the balloon catheter within the guiding catheter during the angioplasty procedure. The transition region is the region from which the guidewire emerges from the balloon catheter at the proximal guidewire port, and it was found to be important to maintain this region within the guiding catheter during the procedure. Accordingly, the balloon catheter was dimensioned such that the distance between the distal end of the balloon catheter and the transition region and/or the proximal guidewire port was at least 10 cm. However, such a dimension has been found to be problematic for several reasons. For catheters which are designed for stent delivery, the distal portion of the catheter in the vicinity of the balloon and proximal thereof should be simultaneously very flexible to navigate the coronary arteries, have good column strength to provide pushability, and have good kink resistance. By comparison, the remaining, proximal portion of the catheter generally requires good column strength and less flexibility. Flexibility of the distal portion of the catheter is important for maneuverability and deliverability of the catheter. However, flexibility must be balanced so as not to compromise the ability of the catheter to track over the guidewire, or permit bowing or looping of the catheter or other movement of the catheter away from the guidewire, resulting in loss of pushability. Extending the distance between the end of the catheter and the proximal guidewire port detracts from some of these desired characteristics, for example by reducing pushability.

Another issue relating to the flexibility of the distal portion of the catheter is due to the composition of the catheter. Known rapid exchange balloon catheters and over-the-wire balloon catheters are formed by blowing a balloon from a length of suitable tubing, trimming the ends of the tubing close to the balloon and bonding the balloon onto a catheter shaft. However, the bond between the balloon and the shaft at a location close to the balloon proximal end forms a thickened and stiffer portion at a location where high flexibility and trackability are desired.

### SUMMARY OF THE INVENTION

In accordance with the invention, the afore-mentioned deficiencies in the prior art are addressed by providing a stent delivery balloon catheter including a balloon leg having an extended length so that the bonding junction between the balloon and the catheter shaft is move proximally. In addition, a distance between a proximal guidewire ports and the distal end of the catheter is reduced from traditional rapid exchange catheters to achieve a move rapid termed super rapid exchange. In this manner, pushability of the distal portion of the balloon catheter and trackability over the guidewire are improved, while at the same time more rapid exchanged are possible. Improved pushability of the distal portion of the balloon catheter and trackability over the guidewire, and reduced possibilities of shaft bowing away from the guidewire, or guidewire looping can also be achieved by providing a stiffening wire, having several possible configurations, that extends at least partially along the length of the balloon leg and catheter shaft. In addition, to provide lesion dimension measurements, radiopaque marker bands are provided on the guidewire tube such that a visual frame of reference is available against which lesion measurements can be made in order to assist with proper stent or balloon selection.

### BRIEF DESCRIPTION OF THE DRAWINGS

The invention of the present application will now be described in more detail with reference to embodiments of the apparatus and method exemplifying principles of the invention, given only by way of example, and with reference to the accompanying drawings, in which:

FIG. 1 is a cross-sectional view of a distal portion of a stent delivery catheter;

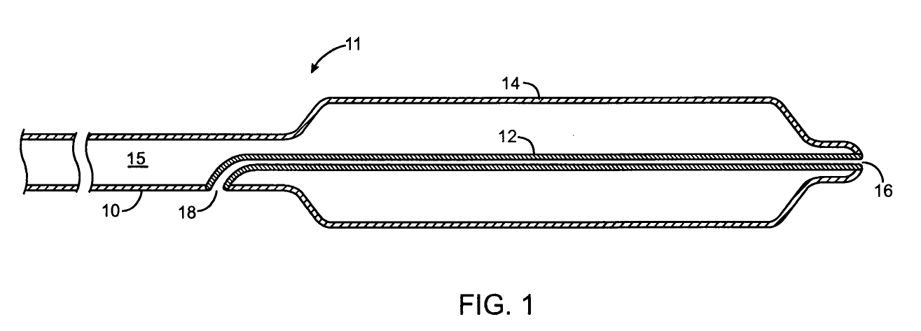

FIG. 2 is a schematic view showing placement of a stent in a vascular lesion during a stent delivery procedure;

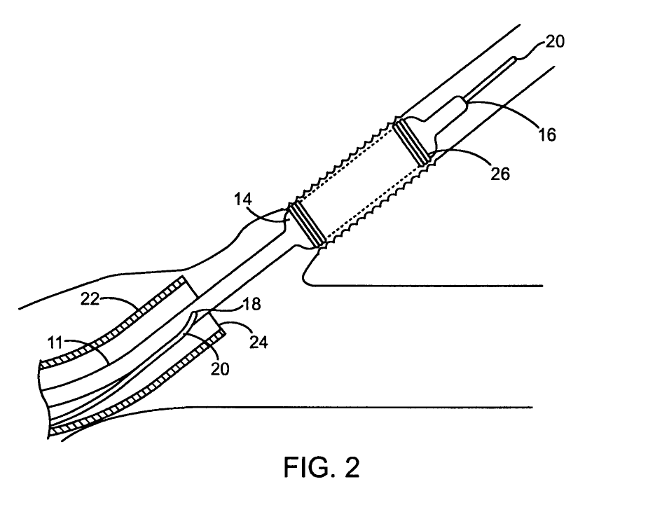

FIG. 3 is a schematic view of a rapid exchange stent delivery assembly;

FIG. 4 is a cross-sectional view of a distal portion of a rapid exchange stent delivery catheter;

FIG. 5 is a more detailed cross-sectional view of the distal portion of FIG. 4 showing the balloon leg and connection region;

FIG. 6 is a cross-sectional view showing a stiffening wire;

FIG. 6A is a more detailed view of the stiffing wire;

FIG. 7 is a cross-sectional view showing the use of marker bands; and

FIG. 8 is a cross-sectional schematic view showing the marker bands in use against a lesion.

### DETAILED DESCRIPTION OF EXEMPLARY EMBODIMENTS

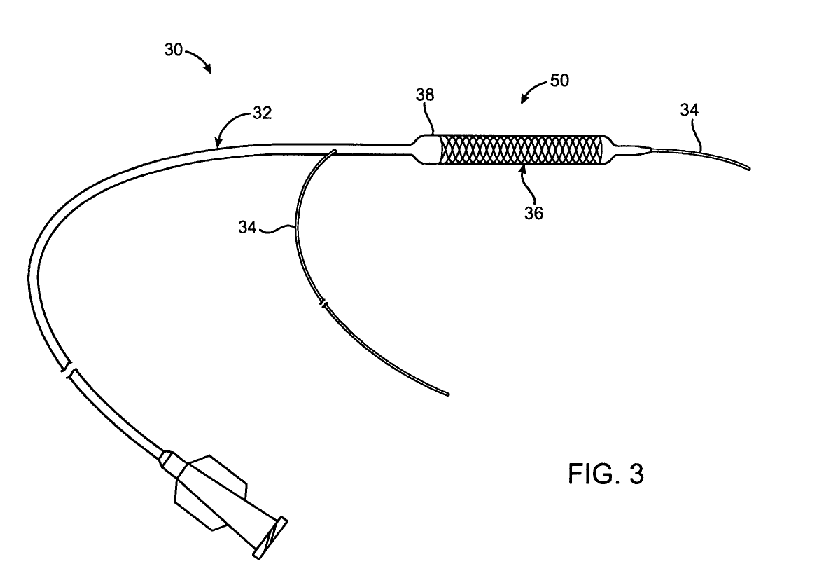

FIG. 3 shows an assembly 30 including a rapid exchange stent delivery catheter 32, a guidewire 34, and a stent 36. The stent 36 is seated over an expandable portion 38 of a balloon 50 disposed in the distal portion of the catheter. The stent 36 is shown in an expanded state. In the unexpanded or delivery configuration, the expandable portion 38 of the balloon 50 and stent 36 will have an outer diameter close to the outer diameter of the shaft of the catheter 32 for easy maneuverability through the patient's vasculature.

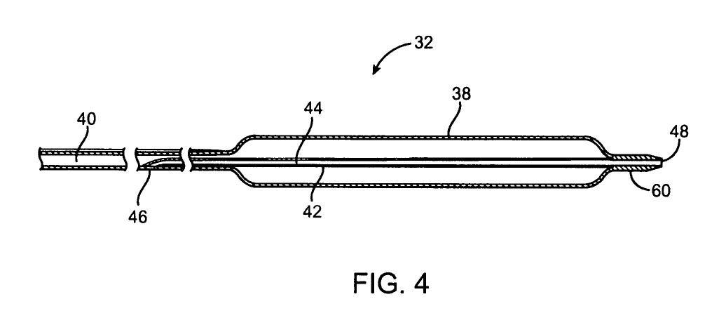

As seen in FIG. 4, the expandable portion 38 is in fluid communication with a primary lumen 40 of the stent delivery catheter 32 defined by the generally tubular nature of the catheter shaft over substantially its entire length. The primary lumen 40 serves to deliver fluid, such as saline, to and from the expandable portion 38 to inflate or deflate the balloon. A secondary, guidewire tube 42 defining a secondary lumen 44 through which a guidewire (not shown) passes is disposed within the catheter 32. Secondary tube 42 extends through the interior of expandable portion 38 and is bonded to the catheter 32 at both ends of the secondary tube. Secondary lumen 44 of secondary tube 42 is in communication with the exterior of the catheter 32 at a proximal guidewire port 46 and a distal guidewire port 48.

In order to control the flexibility and maneuverability of the stent delivery catheter 32, portions of the shaft of the catheter may advantageously include a structure or features that alter its structure and behavior in response to forces exerted during manipulation. According to an advantageous embodiment of the present invention, a portion of the shaft includes one or more lengths of hypotubing, one or more overlayers or exterior jackets, or both. Further, the hypotubing may include features that modify its flexibility characteristics, such as suitably-oriented (circumferential, longitudinal, helical, etc.) and pitched cuts or grooves or other modifications to its structure that commensurately change its flexibility characteristics, particularly at a distal portion of the catheter in the vicinity of the expandable portion 38, where increased flexibility may be desired.

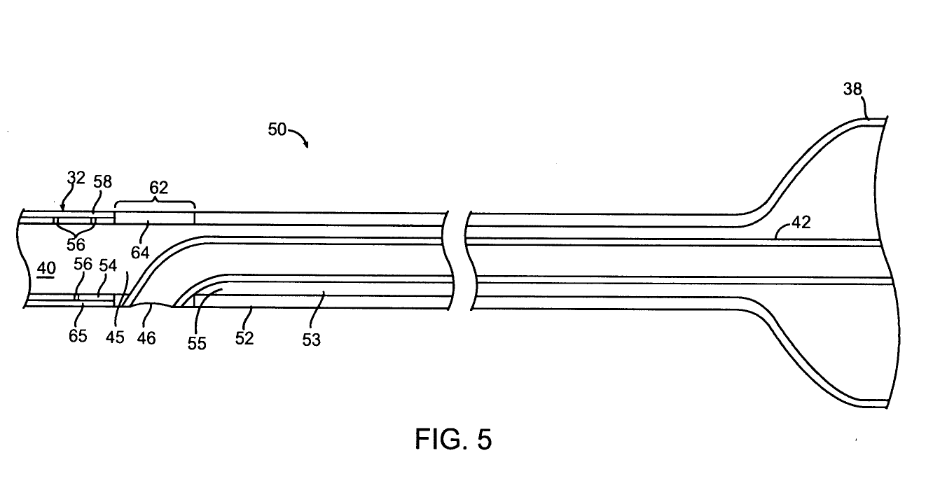

FIG. 5 illustrates a cross-sectional view of a distal portion of the stent delivery catheter 32 according to an embodiment of the invention. The expandable portion 38 is formed as part of a balloon 50, which balloon also includes a proximal balloon leg 52 extending in the proximal direction from the expandable portion 38 and defining a balloon leg lumen 53. Between the expandable portion 38 and the balloon leg 52 is the balloon proximal shoulder. Unlike expandable portion 38, balloon leg 52 is not expandable, but rather maintains its shape and dimensions under operating pressures. The length of balloon leg 52 is at least about 2 cm measured from the edge of the balloon proximal sholder (?) The catheter 32 includes features such as hypotube 54 and helical cuts 56 formed in the hypotube to thereby modify the flexibility characteristics of the distal portion of the catheter. An outer polymer jacket 58 surrounds hypotube 54. By forming the jacket 58 of a biocompatible material, e.g., a biocompatible polymer, the catheter 32 can be made fluid tight to the inflation fluid passing through primary lumen 40, relatively low friction to assist in passing the catheter 32 through the vasculature of a patient, and the flexibility of the catheter can further be modified.

Expandable portion 38 is depicted in an inflated or expanded state in FIG. 5. Expandable portion 38 is inflated or expanded by injecting fluid to its interior region. As explained above, balloon leg 52 is structured to essentially retain its shape and dimensions irrespective of the inflation state of expandable portion 38, to the extent permissible. In other words, balloon leg 52 is not intended to be inflatable in the sense that balloon expandable portion 38 is, even though it is preferably made of the same material as the expandable portion and is made as a unitary piece integral therewith. This difference in behavior can be achieved by providing a different thickness profile for the balloon leg 52 than for the expandable portion 38. A greater thickness reduces deformability, making the balloon leg 52 less likely to inflate under fluid pressures that would inflate expandable portion 38. Proximal balloon leg 52, along with a shorter distal balloon leg on the opposite side of expandable portion 38, are formed by a balloon blowing process within a mold in which the mold ensures that the kg portions do not expand when the expandable portion 38 is formed. A distal tip portion 60 of a different (more flexible) material than balloon 50 is bonded to the distal balloon leg.

Balloon 50 is coupled to the remainder of catheter 32 at a connection region 62 by way of a sleeve 64 providing a strong, fluid-tight seal between balloon leg 52 and a shaft 65 of catheter 32. Sleeve 64 couples the distal opening 45 of primary lumen 40 with the proximal opening 55 of balloon leg lumen 53. This coupling provides a bonding region between the proximal leg 52, secondary tube 42 and catheter shaft 65. Sleeve 64 also provides reinforcement for the connection point of secondary guidewire tube 42 in the vicinity of proximal guidewire port 46. Secondary guidewire tube 42 extends at least partially through balloon leg portion 52. Alternatively, coupling of balloon 50 to the remainder of the balloon catheter 32 may be achieved by overlying balloon leg 52 over the shaft 65 of the balloon catheter and bonding these two components together, either by heat shrinking and/or melting, adhesive, or other expedients.

The connection region 62 includes several components, such as sleeve 64 and a proximal portion of secondary guidewire tube 42. The presence of different catheter components at this connection region 62 impacts the flexibility characteristics of this portion of the catheter. Specifically, the additional structures detract from the flexibility of the catheter 32 in that region. Moving the connection region to a distance of at least 2 cm from the balloon shoulder improves flexibility of the catheter distal end providing improved performance.

Balloon leg 52 is provided in order to distance the connection region 62 from expandable portion 38, since the flexibility of the balloon leg can be better managed than that of the multi-layer connection region.

Thus optimally, the distance from distal the end of the catheter, and specifically from distal guidewire port 48, to proximal guidewire port 46 is preferably in the range of about 5 cm to about 8 cm, and more preferably, is about 7 to about 8 cm. Selection of the materials and structure of balloon leg 52 takes into account the desired flexibility of this portion of the catheter. Typically, the flexibility of the balloon leg 52 of the catheter 32 should be greater than that of shaft 65, and even of the distal portion of shaft 65, which may be more flexible than the proximal portion of the shaft. Suitable materials for the balloon 50 and balloon leg 52, taking these flexibility requirements into account, include, but are not limited to, known balloon materials such as nylon 12.

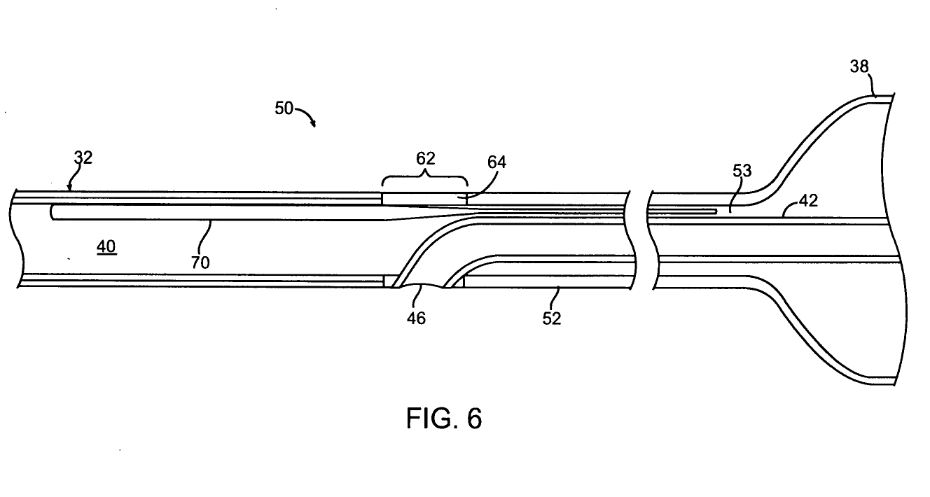

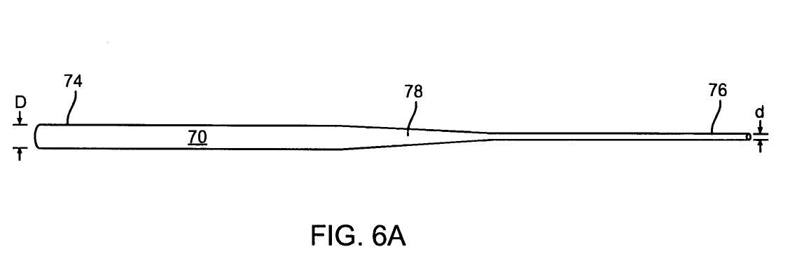

In order to further modify or control the flexibility of the region of catheter 32 proximal to expandable portion 38, a stiffening wire may be provided. With reference to FIG. 6, a stiffening wire 70 is shown disposed longitudinally within catheter 32. Stiffening wire 70, also shown in greater detail in FIG. 6A, has a proximal portion 74, a distal portion 76, and a transition portion 78. Transition portion 78 is in the form of a gradual inward taper from proximal portion 74 to distal portion 76. The distal portion 76 is narrower and more flexible than the proximal portion 74. The diameter d of distal portion 76 is about one half or less of the diameter D of proximal portion 74. Materials from which stiffening wire 70 may be constructed include, but are not limited to, stainless steel, NiTi, and CoCr.

Stiffening wire 70 is preferably free-floating at its ends, and is only attached to the catheter 32 in a region between the two ends, preferably in connection region 62. A preferred attachment method is by way of sleeve 64 to which stiffening wire 70 is bonded. Bonding can be achieved by placing a small polymer sleeve over the wire 70 and bonding the small polymer sleeve into the connection region 62. Wire 70 extends at least partially into lumen 53 of balloon leg 52, and preferably into the entire length of the lumen 53, but ends proximally of the balloon proximal shoulder. Wire 70 also extends proximally at least partially into lumen 40 of shaft 65. An advantage of stiffening wire 70 is that it improves trackability over the guidewire by providing resistance to bending to both balloon leg 52 and shaft 65 at the shaft's distal and most flexible portion. This compensates for the increased length provided by balloon leg 52 and required to maintain the connection region 62 and proximal guidewire port 46 within the guiding catheter during stent delivery. The stiffening wire 70 thus can reduce bowing of the shaft 65 of the catheter 32 away from the guidewire, kinking of the catheter or looping of the guidewire. In addition, the increase in length afforded by the balloon leg 52 is limited in order to avoid introducing too much flexibility and pushability problems.

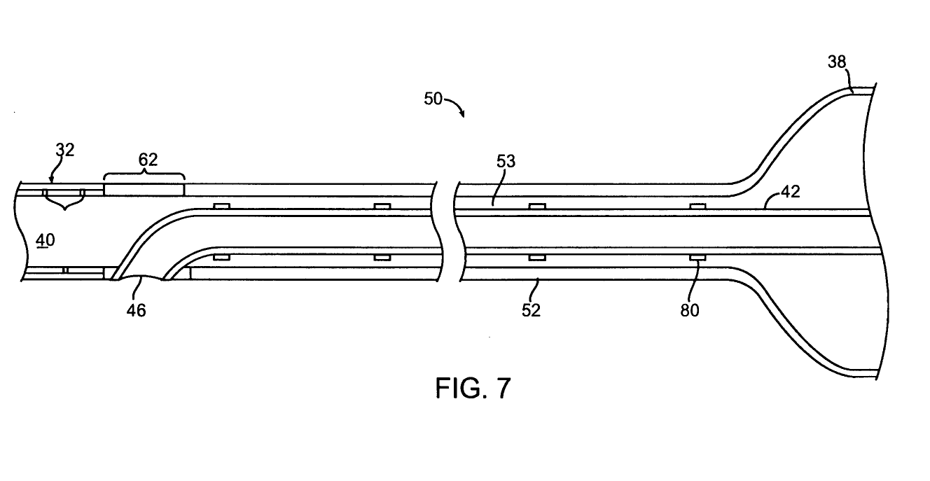

Another advantageous feature that can be provided with catheter 32 can be referred as a radiopaque measuring stick and is described with reference to FIG. 7. Three or more radiopaque marker bands 80 are disposed in balloon leg 52, preferably annularly around secondary guidewire tube 42. The maker bands 80 are evenly spaced apart, for example every 1 cm or every 5 mm measured center-to-center. The length of each marker band 60 is about 0.1 to about 1.0 mm. Use of marker bands in this configuration provides a visual indication of the length of a lesion or other physiological feature of the patient against which is useful in situations where length is difficult to determine. Suitable radiopaque materials for the marker bands 60 include, but are not limited to, barium, platinum, iridium, gold or combinations thereof.

In use, the expandable portion 38 is passed beyond the physiological feature to be measured and the measuring stick is aligned within the feature. A lesion that is 3 cm long for example would extend over three marker bands spaced 1 cm apart, enabling a physician to select a suitably dimensioned stent based on such a determination, or to determine if a selected stent is of an appropriate size.

Although the foregoing describes aspects of the present invention in the context of a rapid exchange stent delivery catheter, the present invention is not limited to such devices. Accordingly, additional embodiments exemplifying principles of the present invention include rapid exchange and non-rapid exchange catheters, balloon and non-balloon catheters including, but not limited to, infusion catheters, angioplasty catheters, angiography catheters, thermal and/or RF and/or laser ablation catheters, and fixed-wire vascular catheters.

With reference to the drawing figures, an exemplary method of stent implantation embodying further principles of the present invention will now be described. Preferably a stenosed region of a blood vessel a mammalian, preferably human, patient is first predilated with an angioplasty balloon catheter. A catheter in accordance with the present invention is then inserted into the vasculature optionally over a guidewire, and is advanced through a guide catheter to a vascular location of interest. The balloon of the catheter may then be inflated or expanded in a manner well appreciated by the skilled artisan, for example by increasing the pressure applied to an inflation fluid, and the balloon's diameter increases. When a stent is positioned on the exterior surface of the balloon, the stent is thus expanded, in a well know manner. Thus, the balloon and/or the stent can be expanded against the interior surface of the blood vessel. Due to the relative short rapid exchange length of about 8 cm or less the proximal guidewire port 46 may be positioned outside of the guide catheter during a procedure.

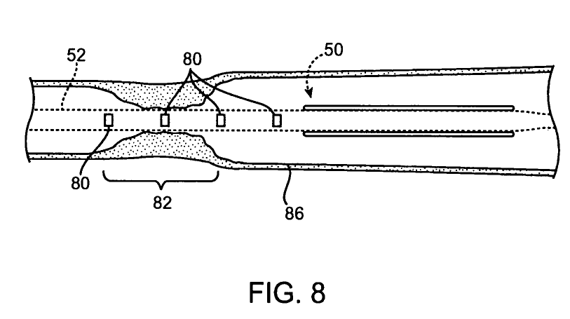

With reference to FIG. 8, a procedure for determining the size of a lesion, for example in order to select an appropriate stent size balloon size, or determine whether a selected stent is appropriate, is described. The balloon 50 of catheter 32 is shown in the vicinity of lesion 82 of vessel 86. Balloon 50 is introduced into this vicinity in the manner described above. Proximal balloon leg 52 is disposed such that radiopaque marker bands 80 can be visualized inside the lesion 82, and the dimensions of the lesion 82 can be compared with the position and number of marker bands 88. In the illustrated example, the length dimension of the lesion 82 corresponds to about three marker bands 88, making its length about 1 cm assuming 0.5 cm separation between marker bands. This can assist the physician to determine whether the stent selected is of a correct size. If the stent is not of a correct size, it can be withdrawn and a more suitable stent the lesion can then be selected.

While the invention has been described in detail with reference to exemplary embodiments thereof, it will be apparent to one skilled in the art that various changes can be made, and equivalents employed, without departing from the scope of the invention. Each of the aforementioned documents is incorporated by reference herein in its entirety.

What is claimed is:

1. A rapid exchange stent delivery catheter comprising:
a catheter shaft defining a primary lumen;
a balloon including an expandable portion and a balloon leg that are in fluid communication with the primary lumen; and
a guidewire exchange tube extending through the expandable portion and at least partially through the balloon leg, the guidewire exchange tube defining a secondary lumen communicating with the exterior of the catheter at proximal and distal guidewire ports separated by a distance in the range of about 5 cm to about 8 cm.

2. The rapid exchange stent delivery catheter of claim 1, further wherein the proximal and distal guidewire ports are separated by a distance of about 7 to about 8 cm.

3. The rapid exchange stent delivery catheter of claim 1, wherein the balloon leg is more flexible than the catheter shaft.

4. The rapid exchange stent delivery catheter of claim 1, wherein the balloon leg is of the same material as the expandable portion.

5. The rapid exchange stent delivery catheter of claim 1, wherein the balloon leg is integral with the expandable portion.

6. The rapid exchange stent delivery catheter of claim 1, wherein the balloon leg at least about 2 cm in length.

7. The rapid exchange stent delivery catheter of claim 1, further comprising:
a stent on the exterior of the expandable portion.

8. The rapid exchange stent delivery catheter of claim 1, further comprising a stiffening wire that has at least a portion that extends longitudinally at least partially into the balloon leg.

9. The rapid exchange stent delivery catheter of claim 8, wherein the stiffening wire has at least a portion that extends longitudinally at least partially into the catheter shaft.

10. The rapid exchange stent delivery catheter of claim 9, wherein the stiffening wire is circular in cross section, and wherein the portion extending at least partially into the balloon leg has a diameter that is smaller than the diameter of the portion extending at least partially into the catheter shaft.

11. The rapid exchange stent delivery catheter of claim 8, wherein the stiffening wire has free-floating ends.

12. The rapid exchange stent delivery catheter of claim 8, wherein the stiffening wire does not extend into the expandable portion.

13. The rapid exchange stent delivery catheter of claim 8, further comprising a sleeve coupling the balloon leg to the catheter shaft, said stiffening wire being bonded exclusively to said sleeve.

14. The rapid exchange stent delivery catheter of claim 13, wherein the guidewire exchange tube is connected to the sleeve at the proximal guidewire port.

15. The rapid exchange stent delivery catheter of claim 1, further comprising a plurality of evenly-spaced marker bands provided in the balloon leg.

16. The rapid exchange stent delivery catheter of claim 15, wherein the marker bands are disposed annularly around the guidewire exchange tube.

17. The rapid exchange stent delivery catheter of claim 1, further comprising a stent seated on the expandable portion of the balloon.

18. A rapid exchange stent delivery catheter comprising:
a catheter shaft defining a primary lumen having a distal opening;
a balloon in fluid communication with the primary lumen, the balloon including an expandable portion and a non-expandable balloon leg integral with the expandable portion, the balloon leg having a length of at least about 2 cm; and
a guidewire exchange tube extending through the expandable portion and at least partially through the balloon leg, the guidewire exchange tube defining a secondary lumen communicating with the exterior of the catheter at a distal guide wire port at a distal end of the catheter and at a proximal guidewire port disposed at least about 5 cm and less than about 25 cm from the distal guidewire port.

19. The rapid exchange stent delivery catheter of claim 18, further comprising a sleeve coupling said distal opening with said proximal opening.

20. The rapid exchange stent delivery catheter of claim 19, wherein said proximal guidewire port is disposed in said sleeve.

21. The rapid exchange stent delivery catheter of claim 18, wherein said proximal and distal guidewire ports are separated by a distance in the range of about 5 cm to about 8 cm.

22. The rapid exchange stent delivery catheter of claim 18, wherein the proximal and distal guidewire ports are separated by a distance of about 7 to about 8 cm.

23. The rapid exchange stent delivery catheter of claim 18, wherein the balloon leg is more flexible than the catheter shaft.

24. The rapid exchange stent delivery catheter of claim 18, wherein the balloon leg is integral with the expandable portion.

25. The rapid exchange stent delivery catheter of claim 18, wherein the balloon leg at least about 2 cm in length.

26. The rapid exchange stent delivery catheter of claim 18, further comprising a stiffening wire that has at least a portion that extends longitudinally at least partially into the balloon leg lumen.

27. The rapid exchange stent delivery catheter of claim 26, wherein the stiffening wire has at least a portion that extends longitudinally at least partially into the catheter shaft.

28. The rapid exchange stent delivery catheter of claim 27, wherein the stiffening wire has free-floating ends.

29. The rapid exchange stent delivery catheter of claim 19, further comprising a stiffening wire bonded exclusively to said sleeve.

30. The rapid exchange stent delivery catheter of claim 18, further comprising a plurality of evenly-spaced marker bands provided in the balloon leg.

31. The rapid exchange stent delivery catheter of claim 30, wherein the marker bands are disposed annularly around the guidewire exchange tube.

32. The rapid exchange stent delivery catheter of claim 18, further comprising a stent seated on the expandable portion of the balloon.

33. A rapid exchange stent delivery catheter comprising:
a catheter shaft defining a primary lumen;
a balloon including an expandable portion and an integral balloon leg that are in fluid communication with the primary lumen;
a guidewire exchange tube extending through the expandable portion and at least partially through the balloon leg, the guidewire exchange tube defining a secondary lumen communicating with the exterior of the catheter at proximal and distal guidewire ports; and
a stiffening wire having free-floating ends, the stiffening wire being disposed at least partially in the primary lumen of the catheter shaft and being bonded to a sleeve disposed between the catheter shaft and the balloon leg.

34. The rapid exchange stent delivery catheter of claim 41, wherein the stiffening wire extends into a balloon leg lumen defined by the balloon leg.

35. The rapid exchange stent delivery catheter of claim 41, wherein the balloon leg is integral with the expandable portion.

36. The rapid exchange stent delivery catheter of claim 41, wherein the balloon leg at least about 2 cm in length.

37. The rapid exchange stent delivery catheter of claim 41, further comprising a stent seated on the expandable portion of the balloon.

38. A method of performing a vascular procedure in a mammalian patient, comprising:
inserting a catheter into the vasculature of the patient, the catheter including
a catheter shaft defining a primary lumen;
a balloon including an expandable portion and a balloon leg that are in fluid communication with the primary lumen; and
a guidewire exchange tube extending through the expandable portion and at least partially through the balloon leg, the guidewire exchange tube defining a secondary lumen communicating with the exterior of the catheter at proximal and distal guidewire ports separated by a distance in the range of about 5 cm to about 8 cm, said inserting including passing the catheter over a guidewire extending disposed in the secondary lumen; and
advancing the catheter to a location in the vasculature of the patient.

39. The method of claim 46, further comprising a stent seated on the expandable portion of the balloon.

40. The method of claim 48, further comprising a stent seated on the expandable portion of the balloon.
